(19) United States
(12) Patent Application Publication
(10) Pub. No.: US 2009/0259305 A1
(43) Pub. Date: Oct. 15, 2009

(54) Implantable Prosthetic Holder and Handle

(76) Inventors: Fred Lane, Mosinee, WI (US); Donald Grover, Rothschild, WI (US)

Correspondence Address: LANE PATENTS LLC, 100 NORTH 72ND AVE., SUITE 107, WAUSAU, WI, 54401, US

(21) Appl. No.: 12/422,797

(22) Filed: Apr. 13, 2009

Related U.S. Application Data
(60) Provisional application No. 61/044,483, filed on Apr. 12, 2008.

(51) Int. Cl. A61F 2/24

(52) U.S. Cl. 623/2.11

(57) ABSTRACT

An improved implantation holder apparatus and system for holding bioprosthetic aortic valves for insertion into a heart. The handle system interacts with implantation holder and the valve in such a way that commissure support struts may be retracted by simply rotating the handle which allows the surgeon more room to work in tight quarters to complete the implantation process. Included is a handle system, which can be attached and detached, for securing the holder during application of the parachute stitching and moving the valve into position. Once the valve is in position, the handle may be retained in place, or removed for greater ease of access to the aortic cavity. Once the valve is fully sutured into place the handle may be easily reattached to the holder with an automatic cutting system to cut the sutures and allow the holder to be more easily and safely be retracted from the aorta.

### CROSS REFERENCE TO RELATED APPLICATIONS

This application claims priority from U.S. Provisional Application No. 61/044,483 filed 12 Apr. 2008; and is incorporated herein by reference in its entirety.

### BACKGROUND OF THE INVENTION

1. Field of the Invention

The present invention relates to the field of valvular prostheses, and, more particularly, to a bioprosthetic heart valve holding tool and system for implantation of aortic valves which retracts the struts during implantation and is minimally intrusive.

2. Description of the Related Art

The relevant art of interest describes various devices for implanting an aortic valve, in particular.

Many surgeons look to streamline the process of valve replacement. The process is quite invasive on the patient. The procedure for implantation is risky with the risk being increased the longer the procedure takes. The surgeon also views every point of the procedure as a possible point of error. The surgeon is continually looking to remove steps, minimize time, and eliminate possible points of error. In addition, access to the aorta by the surgeon requires working in tight quarters in best of conditions allowing little room to maneuver. Therefore, the surgeon requires a minimally invasive methodology, which allows the procedure to be completed with a least number of meaningful steps.

U.S. Pat. No. 7,189,258 B2 issued to Johnson et al Mar. 13, 2007; discloses the current valve and holder. It discloses a methodology built upon a thread and reel apparatus. This methodology, as a broad approach, dates back to at least 1979. This system has several drawbacks. Firstly; reeling of the spool, without also rotating the heart valve, requires that an opposing member be placed to keep the valve from rotating. This is accomplished by running a series of legs between the commissure posts and securing them with a suture. The suture at the bottom of the leg then acts as an anti-rotation member which places strain on that portion of the stent and accompanying material, which is not ideally desired. Further, many surgeons find in practice that their knots required to suture the prosthetic in place catch in the leg stent union due partially to the tight working quarters. This is a potentially dangerous result, as loosely, poorly, or improperly tied sutures can lead to separation or partial separation of the stent and further lead to paravalvular leakage. In addition, the commissure support strut is also reeled in by a suture which is placed into the tissue at the apex of the strut, thus pulling on the prosthetic to retract the strut. This stress to the prosthetic is deemed undesirable. When the surgeon is finished with the process of implantation of the prosthetic, and in order to remove the holder, the surgeon is required to enter the cavity with a knife to remove the suture. This further risks the patient for being poked or cut with the knife.

UK patent number GB 2 108 393 issued to Extracorporeal Medical Specialties Inc. Published 18 May 1983 and filed in 1979, was the first to disclose the approach upon which the Johnson patent is based.

U.S. Pat. Nos. 5,476,510 and 5,716,401 a child patent issued to Eberhard et al. Dec. 19, 1995 and Feb. 10, 1998 respectively disclose two inventions. First is a high profile valve holder with a line and reel type strut synch. Second is a push structure spread across three levels of strut attachment with cleated detents. The requirement of three levels of attachment makes this apparatus higher profile than is ideally required. The present invention, without this limitation, is much more elegant with a lower profile and all located on one plane.

U.S. Pat. No. 4,865,600 issued to Carpentier et al. Sep. 12, 1989; and assigned to Baxter International Inc. discloses a heart valve holder which can be mounted on the bottom of the valve and rotates to reel in sutures which pull in the struts. This invention is designed for mitral valve work and should not be relevant to any of the present disclosure. It is included to show the prevalence in the art of reel type structures.

U.S. Pat. No. 6,019,790 issued to Holmberg et al. Feb. 1, 2000; discloses a valve holder including a set of jaws which can be pinched or pivoted to close on the structure. It has a high profile, which goes against the objective of the present invention.

### SUMMARY OF THE INVENTION

### SUMMARY

The present invention relates to an implantation holder and system for holding a bioprosthetic aortic valve for insertion into a heart. The handle system interacts with the implantation holder and the valve in such a way that commissure support struts may be retracted by simply pushing the receiving facets attached to each one of the struts in the first two embodiments, or rotating the handle in the third embodiment; which allows the surgeon more room to work in tight quarters to complete the implantation process.

Included is a handle system, which can be attached and detached, for securing the holder during application of the parachute stitching and moving the valve into position. Once the valve is in position, the handle may be retained in place, or removed for greater ease of access to the aortic cavity. The implantation holders are designed to stay shut when the handle is removed to allow the surgeon to work without a handle in the way. Once the valve is fully sutured into place the handle may be easily reattached to the holder with an automatic cutting system to cut the sutures and allow the holder to be more easily retracted from the aorta.

It is therefore an object of the invention to incorporate a system comprising a bioprosthetic valve holder for implanting which allows retraction of commissure support struts.

It is another object of the invention to provide retraction of commissure support struts without unduly tensioning the suture for attaching the holder to the stent.

It is another object of the invention to allow rotational retraction of the commissure support struts without torquing of the stent relative to the handle or implant site.

It is another object of the invention to provide a retracting means for the support strut without a need for a series of legs being attached in the valley between the struts of the prosthesis.

It is another object of the invention that the retraction of the commissure support struts is easily reversible.

It is another object of the invention that a number of intermediate positions of the commissure support strut positions are available.

It is another object of the invention that the holder has a low vertical profile to maximize a surgeon's access to the aorta.

It is another object of the invention to allow the handle to be attached and detached as will during the procedure.

It is another object of the invention to allow a means for cutting an removing sutures which does not require the surgeon to insert a knife into the aortic cavity to remove the sutures which attach the holder to the prosthetic.

These and other advantages of the present invention will become readily apparent upon review of the following specification and drawings.

### BRIEF DESCRIPTION OF THE DRAWINGS

A complete understanding of the present invention may be obtained by reference to the accompanying drawings, when considered in conjunction with the subsequent, detailed description, in which:

FIGS. 1, 2, 3, 4, 5, 6, 7, and 8; denote one embodiment. FIGS. 9, 10, 11, and 12; denote a second embodiment. FIGS. 13, 14, 15 and 16; denote a third embodiment.

FIGS. 1a, b are side perspective and front partial cut away views respectively of a handle member holding an implantation holder with a valve prosthesis;

FIG. 2 is an top exploded view perspective view of an implantation holder comprising a base member and three prosthesis holders aligned above their respective commissure support struts;

FIG. 3 is a top perspective view of an implantation holder having prosthesis holders inserted in a closed position;

FIG. 4 is a perspective view of an implantation holder connected with a valve prosthesis in an open position;

FIGS. 5a, b, c, d, are respectively; top perspective, bottom perspective, side with ghosted through lines, and bottom plan with ghosted through lines, views of a base member for an implantation holder;

FIGS. 6a, b, c, d, e, are respectively; top elevated perspective, bottom front perspective, bottom back perspective, top plan view with ghosted lines, and a sectional view from letter B, of a prosthesis holder;

FIGS. 7a, b, c, d, are respectively; top plan, top perspective, bottom perspective, and a detailed perspective view of a section of the implantation holder, comprised of base member having multiple receiving facets, in an open position;

FIGS. 8a, b, c, d, are respectively; top plan, top perspective, bottom perspective, and detailed perspective views of the implantation holder, comprised of base member having multiple receiving facets, in a closed position;

Supplemental sheets having enlarged versions of FIGS. 7d, 7c, 8d and 8c are provided for further clarification.

FIGS. 9a, b, c d, are respectively; top plan, top perspective, bottom plan and bottom perspective views a second alternate embodiment of the implantation holder.

FIG. 10 is bottom perspective view showing a base member with one of the prosthesis holders in exploded view to show details of the fastening mechanism.

FIG. 11 is a front prospective partial view of a retraction guide with retraction head showing a retraction guide.

FIG. 12 is a bottom plan view of a base member with three prosthesis receiving facets attached in a closed position.

FIG. 13 is a top plan view of a third embodiment of an implantation holder in an open position.

FIG. 14 is a top plan view of a third embodiment of an implantation holder in a closed position.

FIG. 15 is a top perspective view of a base member of a third embodiment.

FIG. 16 is a top perspective view of a prosthesis arm of a third embodiment.

For purposes of clarity and brevity, like elements and components will bear the same designations and numbering throughout the FIGUREs. As the prosthesis holder comprises multiple separate receiving facets of similar structure, these will be designated with alpha characters ‘a, b, and c’ respectively.

| Reference | | |
|---|---|---|
| Number | Element | Descriptors |
| 10 | implantation holder | stiff rouged material - like glass impregnated nylon |
| 12 | valve prosthesis | |
| 14 a, b, c | comissure support | |
| | strut | |
| 16 | prosthesis base | |
| 18 | attachment ring | for sewing into place |
| 20 | supple valves | tissue, urethane, Teflon |
| 30 | base member | structure for prosthesis holder |
| 32 | retraction guide | dado like structure for a guide |
| 34 | retention ledge | mating with retraction guide to hold the prosthesis |
| | | holder with the base member |
| 36 | bottom base | bottom surface for mounting elements |
| 38 | top base | top surface |
| 40 | channel | for receiving a center rail of a prosthesis holder |
| 42 | collar | supporting the handle |
| 44 | collar interface | receiving the handle |
| 60 a, b, c | prosthesis holder | general structure for holding prosthesis |
| 62 a, b, c | receiving facets | interface region between prosthesis and |
| | | commissure support struts |
| 64 | retaining faces | arcurate - mirrored relief of the strut |
| 66 | reciprocating faces | part of the prosthesis holder for mating with the |
| 68 | opposing faces | not used |
| 70 | cleat | pointed ledge, pins for gripping the outside of the |
| | | strut |
| 72 | outer member | centers struts |
| 74 | inner member | holds cleat member and allows rotation |
| 76 | latch | spring like latch for holding the prosthesis holder |
| | | in the closed position |
| 78 | attachment location | |
| 80 | tie | communicates between inner member and outer |
| | | member |
| 82 | retraction rail | support and guide for prosthetic holder retraction |
| 84 | center rail | guide and ‘spring’ for cleat |
| 120 | handle member | |
| 122 | stabilizing collar | |
| 124 | attachment interface | threaded female |
| 128 | attachment interface | male |
| 130 | torquing means | knurled knob |
| 132 | annulus | |
| 133 | bend | |
| 134 | rod | flexible low coeff of friction, free moving |
| 136 | cannular attachment | |
| 138 | gripping surface | cearated |
| 140 | quick release actuator | button |
| 142 | sleeve | |
| 144 | plunger | actuating cutters |
| 210 | implantation holder | second embodiment holder |
| 232 | retraction guide | rectangular box |
| 233 | ratchet button | head mates with ratcheting detents |
| 235 | retention guide | holding retaining facets next to the retraction guide |
| 236 | bottom base | |
| 238 | top base | |
| 242 | collar | supporting the handle |
| 244 | collar interface | receiving the handle |
| 250 | suture holes | for attaching struts to the receiving facets |
| 262 | receiving facets | |
| 264 | retaining faces | |
| 266 | reciprocating faces | |
| 276 | ratcheting detents | holding implantation holder in closed position |
| 277 | ratcheting facets | |
| 282 | retraction rail | |
| 286 | compression faces | mechanically holds the struts |
| 300 | implantation holder | third embodiment |
| 310 | base member | shape sufficient to hold structures |
| 312 | arm pivot | for rotationally mounting arms |
| 314 | center pivot | interface handle to arm |
| 315 | central gear | |
| 316 | engagement member | interfaces with a twist knob on the handle |
| 320 | arm | for expansion and retraction |
| 330 | gears | generally spur gears or comparable |
| 360 | strut button | degree of freedom to rotate |
| 362 | suture holes | for attaching the strut button to the strut |

### DETAILED DESCRIPTION OF THE DRAWINGS

Typically the heart valves that occur in nature are either bicuspid, which comprise two lobes, or valves for regulating blood flow or, more commonly, tricuspid comprising three lobes or valves. While the typical replacement bioprosthetic valve is modeled after a tricuspid system, those skilled in the art will note that this particular system and apparatus can be anticipated for a plurality of lobes.

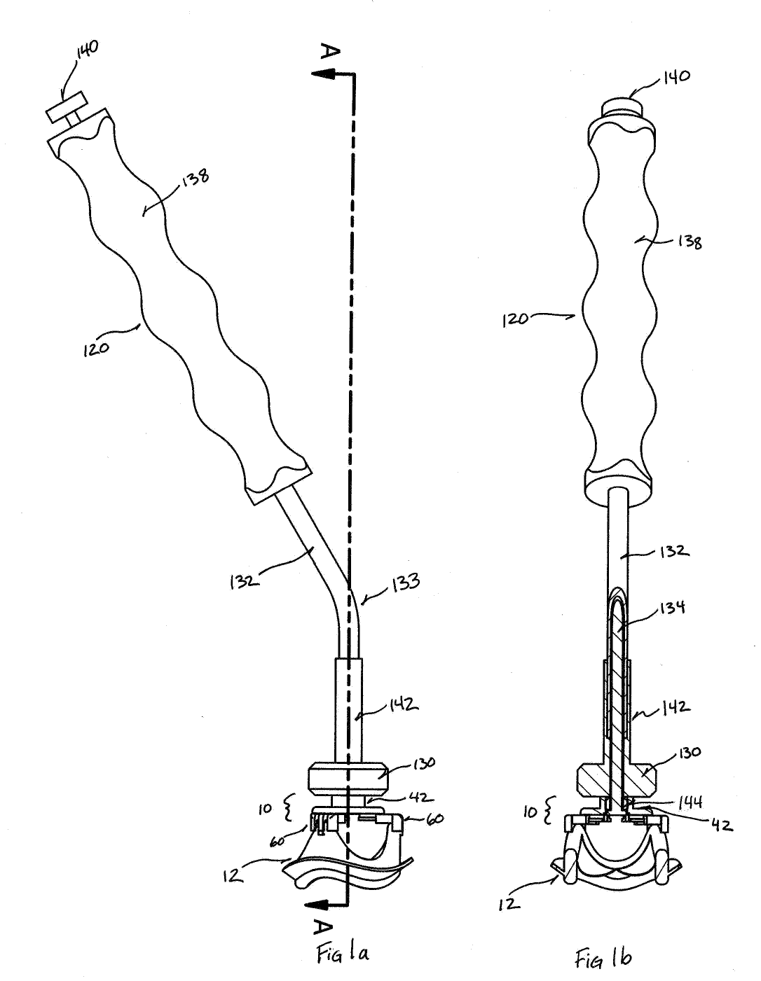

The implantation holder (10), as shown in FIG. 1, with valve prosthesis (12) are positioned into place by a handle member (120), as is shown in FIGS. 1a and 1b. The handle member (120) connects with the implantation holder (10) by means of an annulus (132) having a bend (133) of between 5 and 45 degrees. One skilled in the art may appreciate that the annulus (132) may be comprised of a pliable metal or plastic with plastic memory to allow the user to adjust the angle of the bend (133) at the time of use. A sleeve (142) having a connecting means such as a threaded end or press fit (144) is located at the distal end of the sleeve (142) for the purpose of communicating with the collar (42) of the implantation holder (10) by means of a collar interface (44). A torquing means (130) such as a knurled handle may be added to allow the handle member (120) to be more easily disconnected from the implantation holder (10).

A quick release actuator (140), typically in the form of a button located at the distal end of the gripping surface (138), can communicate with a rod (134) positioned inside the annulus (132) can be used to depress a latch (76), as shown in FIG. 6a, and further detailed in FIG. 8a, causing a quick release of the prosthesis holder (60) from a closed to an open position as illustrated in FIGS. 3 and 4.

### Embodiment One

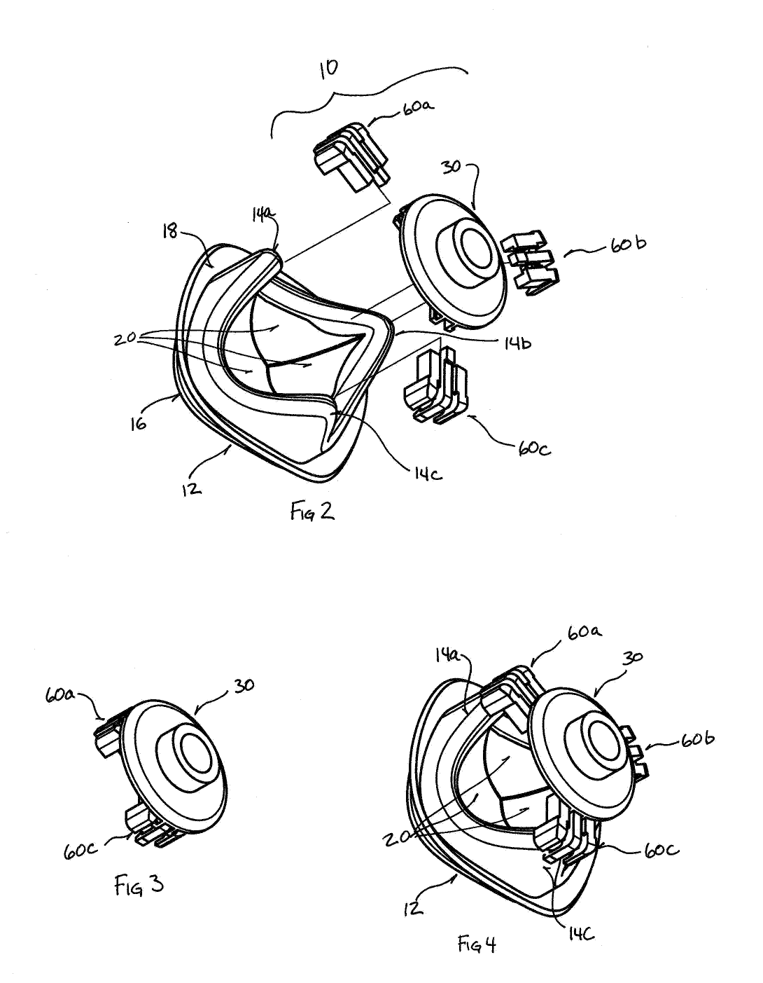

FIG. 2 shows and exploded view of the implantation holder (10) comprising its component parts with a valve prosthesis (12). A typical valve prosthesis (12) is typically comprised of three porcine supple valves 20, but may be of any suitable material of biologic or synthetic origin, such as urethane based polymers, which exhibits the qualities of a human heart valve material. The supple valves (20) are typically mounted to each of a commissure support strut (14a, b, c) such that a one way valve is formed allowing minimal regurgitation between sinus contractions. The valve prosthesis (12) further comprises a prosthesis base (16) for forming the stent or opening for blood flow and can further comprise an attachment ring (18) for attaching, usually by sewing, and sealing the valve prosthesis (12) to the aortic wall.

At least one significant advantage of the implantation holder (10) is shown in the comparison between FIG. 3 which shows the implantation holder (10) in a closed position, and the implantation holder (10) in an open position as shown in FIG. 4. FIG. 4 further shows the valve prosthesis (12), which is not shown in FIG. 3. The closed position comprises a significant reduction in radial area of the commissure support strut (14a, b, c) which allows the surgeon open access to the prosthesis base (16) and the attachment ring (18).

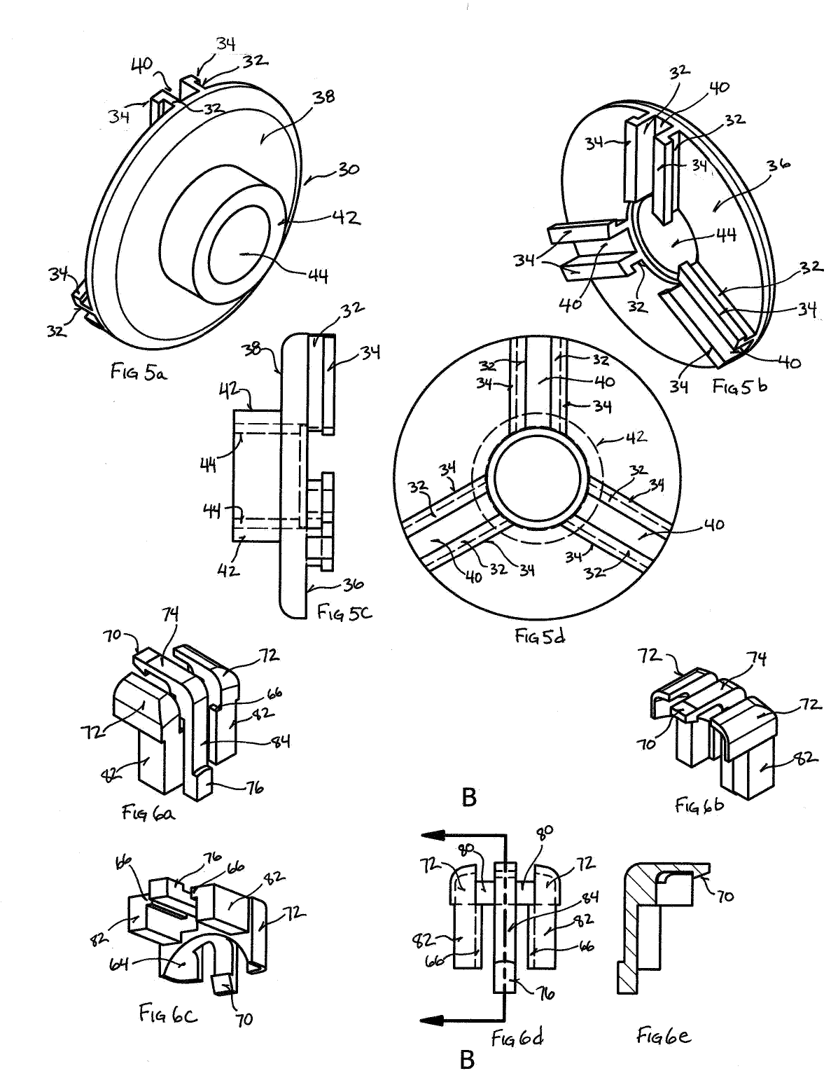

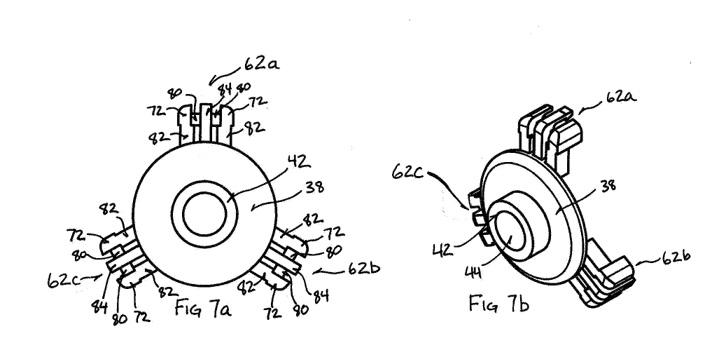

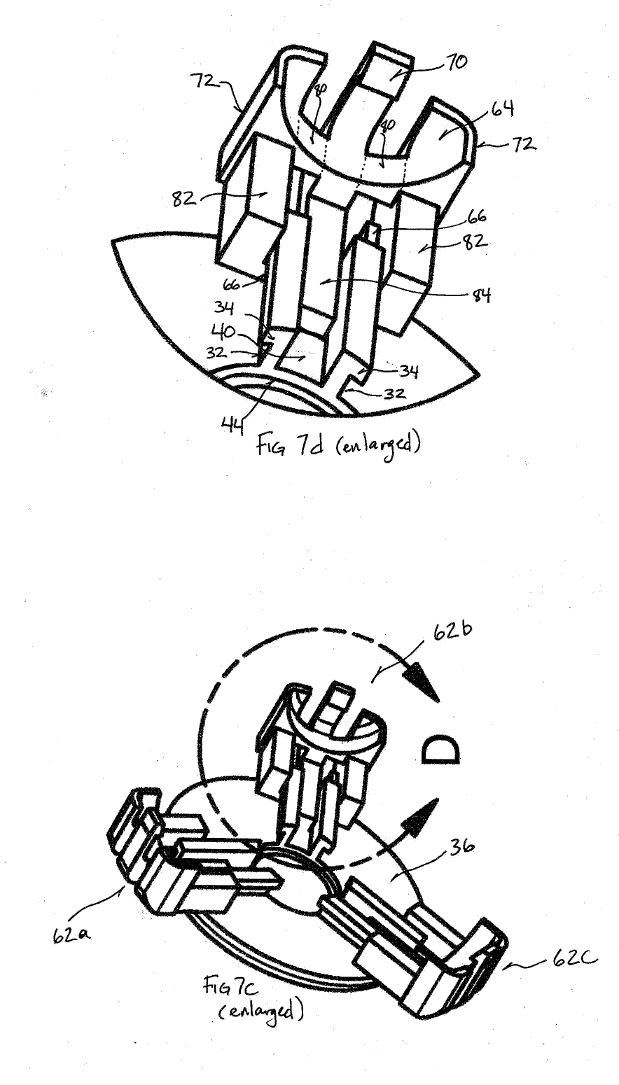

In order to more fully appreciate the advantages of the current system, a more thorough coverage of the implantation holder (10) is required starting with the prosthesis holder (60) shown in FIGS. 5a through 5d. The prosthesis holder (60) is comprised of a plurality of receiving facets (62a, b, c) which conform to, and cradle, the corresponding commissure support strut (14a, b, c). The receiving facets (62a, b, c) are designed such that much of the stress related to retaining sutures, which can be applied in conjunction with the attachment location (78), is relieved during the transition from the open position to the closed position.

The retaining faces (64) can be formed such that the corresponding commissure support strut (14a, b, c) are mirrored for greater stability.

The closing of the prosthesis holder (60) is realized by pressing each of the prosthesis holders (60a, b, c) laterally inward until the latch (76) catches on the inside perimeter of the collar interface (44) giving a predetermined closed diameter for the closed prosthesis holder (60a, b, c) versus an open configuration. A preferred ratio of 1.5 to 1, opened to closed is recommended so as not to overly stress or deform the prosthesis base (16).

Holding of the valve prosthesis (12) may be enhanced by the several retaining faces (66a, b, c) optionally having a cleat (70) which contacts the face of the commissure support strut (14a, b, c) preferably below any tissue area which may be present. The cleat (70) need not be obtrusive and can be formed in a number of embodiments; a cleat, a series of cones, or the like, such that the holding capability of the prosthesis holder (60) is increased.

### Embodiment Two

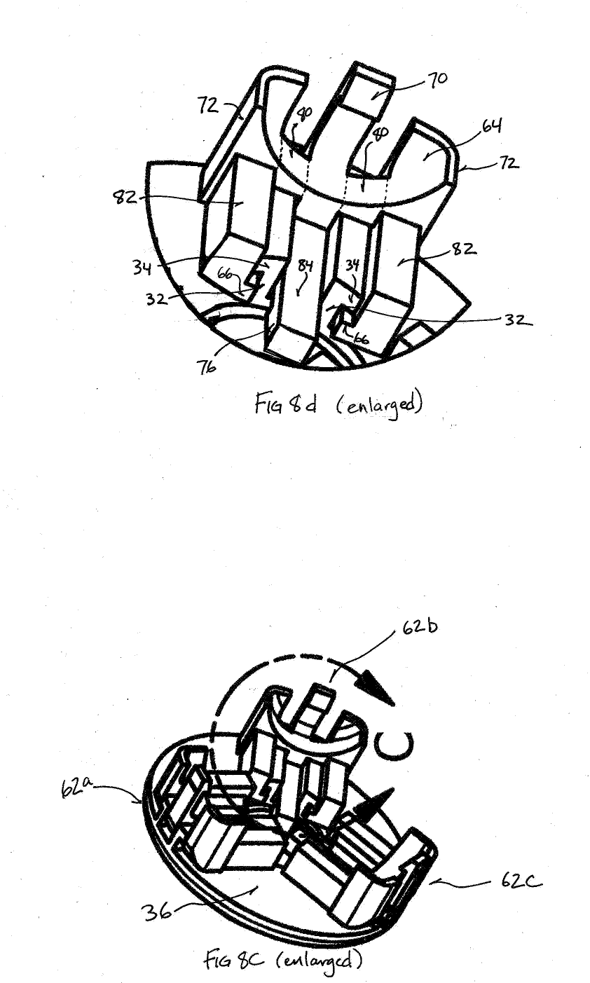

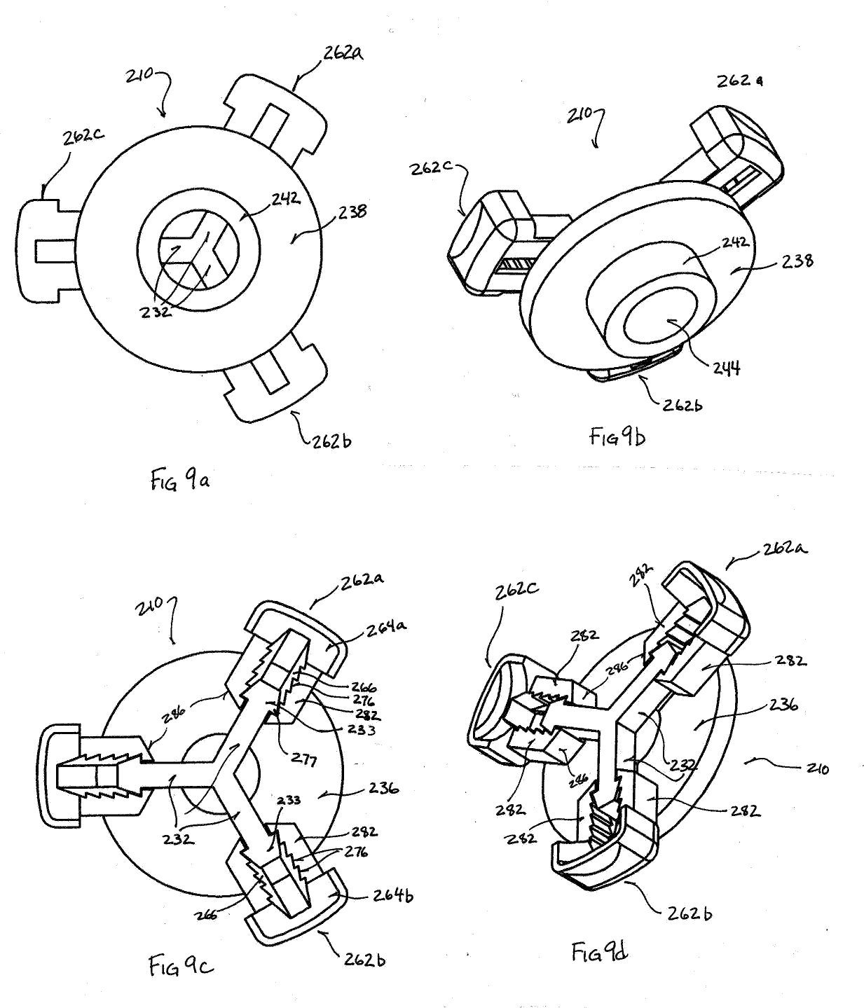

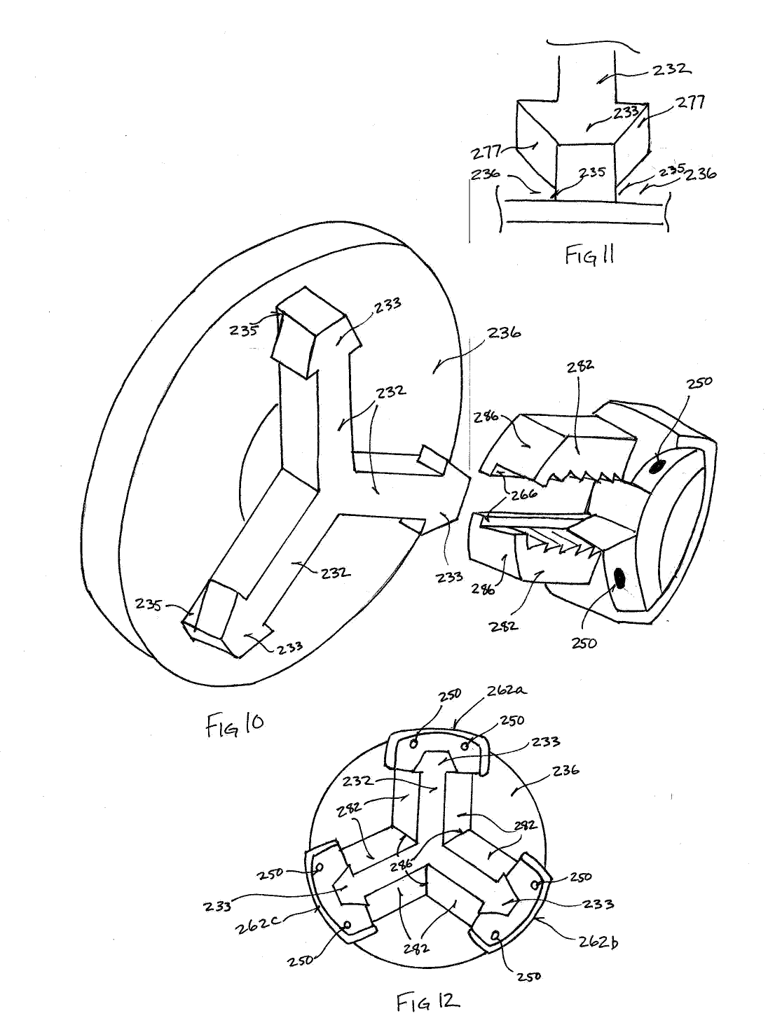

Embodiment two is analogous to embodiment one in operation. FIGS. 9a through 9d and FIGS. 10 and 11, show the implantation holder (210) having three receiving facets (262) being radially dispersed and approximately 120°. The location of the receiving facets (262) being determined by a plurality of retraction guides (232) being attached to the bottom base (236) and having a ratchet button (233) having a retention guide (235) being a vacated trace between the ratchet button (233) and the bottom base (236). The retention guide (235) is matibly fitted with a series of reciprocating faces (266) designed for holding the respective receiving facet (262) to the implantation holder (210). Further the ratchet button (233) comprises a series of ratcheting facets (277) which are interfittably connected with a series of ratcheting detents (276) for providing a plurality of positions between opened and closed.

In the fully closed position, it can be seen that the compression faces (286) matchup quite nicely.

Further, the receiving facets (262) can be provided with a series of suture holes (250) to further secure the corresponding strut (not shown) to the implantation holder (10).

### Embodiment Three

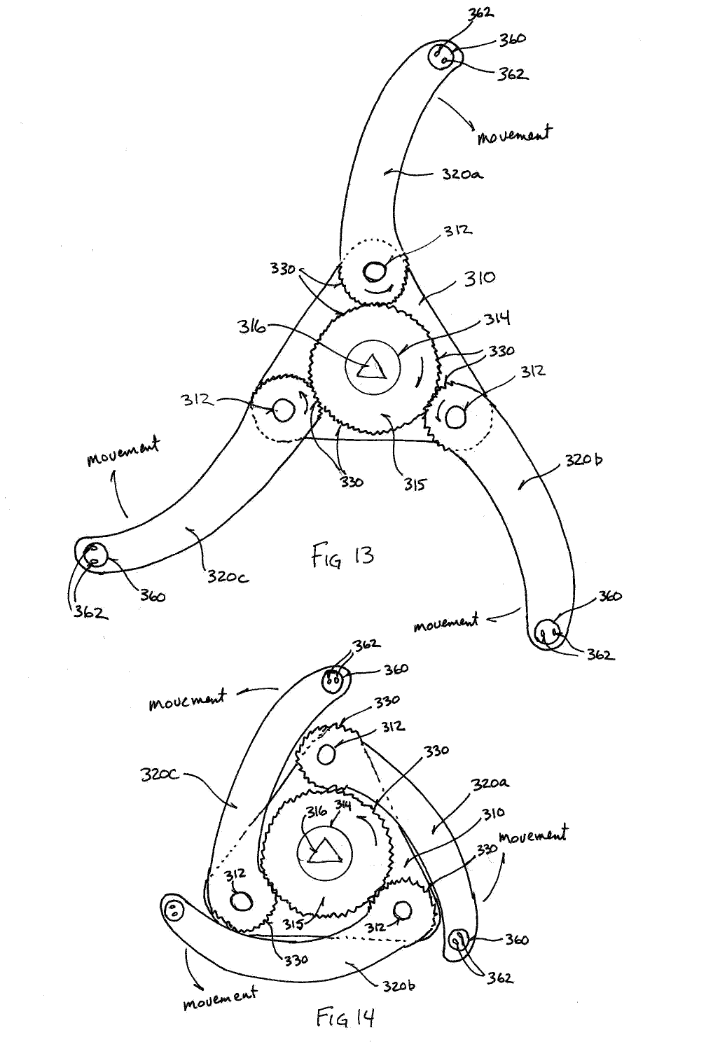

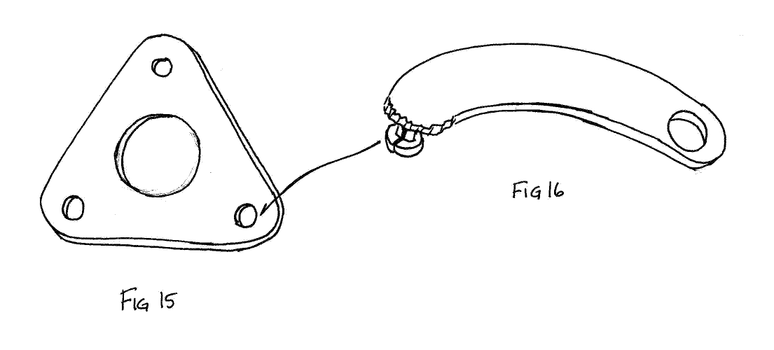

FIGS. 13 through 16 show some aspects of the present embodiment. A base member (310), shown in FIG. 15, sufficient to provide a mounting platform for a series of (312) arm pivots and a center pivot (314) is provided. The base member (310) should be rigid and of rouged construction. A central gear (315) having an engagement member (316), preferably in the form of a triangular void for alignment purposes, and comprising a perimeter of gears, preferably spur gears, however there can be other coupling mechanisms used. A collar mechanism such a provided in the aforementioned embodiments can be provided for attachment to a handle. The central gear (315) is operatively coupled with each of the arms (320) through gears (330) such that rotation of the central gear drives the arms to a position comprising various diameters as shown in comparing FIG. 13 with FIG. 14.

The strut button (360) connects with the corresponding commissure support strut (not shown) through a series of suture holes (362). One skilled in the art can appreciate that the rotation of this mechanism may stress the sutures. Therefore the strut button (360) is rotatably mounted near the distal end of the respective arm (320) to allow stress relief.

### CONCLUSION, RAMIFICATIONS, AND SCOPE

Although the present invention has been described in detail, those skilled in the art will understand that various changes, substitutions, and alterations herein may be made without departing from the spirit and scope of the invention in its broadest form.

Having thus described the invention, what is desired to be protected by Letters Patent is presented in the subsequently appended claims.

What is claimed is:

1. A heart valve clip and handle system for inserting a bioprosthetic heart valve into the heart comprising:
a valve prosthesis comprising a plurality of commissure support struts extending orthogonally from the base of said valve prosthesis;
a prosthesis holder having a plurality of receiving facets being matched with, and removably connected to, said support struts;
a push mechanism being in communication with said receiving facets and having means for retracting said receiving facets which are coupled with said support struts;
a guide for coordinating said means for retracting being integrated with said prosthesis holder, and
a handle member being attached to said guide.

2. The heart valve clip and handle system in accordance with claim 1, wherein said guide comprises an anti-rotation guide.

3. The heart valve clip and handle system in accordance with claim 1, wherein said guide comprises a prosthesis guard.

4. The heart valve clip and handle system in accordance with claim 1, wherein said push mechanism comprises means for retracting having a latch for holding the prosthesis holder in a closed position.

5. The heart valve clip and handle system in accordance with claim 1, wherein said cam knob comprises means for retracting which is reversible.

6. The heart valve clip and handle system in accordance with claim 1, wherein said push mechanism comprises means for retracting having a latch for holding the prosthesis holder comprises a plurality of in a closed positions between open and closed.

7. The heart valve clip and handle system in accordance with claim 1, wherein said handle member is detachable from said guide.

8. The heart valve clip and handle system in accordance with claim 7, wherein said handle member is reattchable to said guide.

9. A heart valve clip and handle system for inserting a bioprosthetic heart valve into the heart comprising:
a valve prosthesis comprising a plurality of commissure support struts extending orthogonally from the base of said valve prosthesis;
a prosthesis holder having a plurality of arms rotatably mounted around a focus being in communication with a gear and having receiving facets being matched with, and removably connected to, said support struts;
a means for actuating the gear mechanism such that the receiving facets move radially outward;
a guide for coordinating said means for retracting being integrated with said prosthesis holder, and
a handle member being attached to said guide.
